(19) United States
(12) Patent Application Publication
(10) Pub. No.: US 2008/0318265 A1
(43) Pub. Date: Dec. 25, 2008

(54) Measuring the Thickness of Organic Samples

(75) Inventor: Francesco Visinoni, Soriole (IT)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Milestone S.r.l., Sorisole (BG) (IT)

(21) Appl. No.: 12/144,927

(22) Filed: Jun. 24, 2008

(30) Foreign Application Priority Data
Jun. 25, 2007 (EP) EP 07 110 951.6

(51) Int. Cl. C12Q 1/02; C12M 1/34

(52) U.S. Cl. 435/29; 435/287.1

(57) ABSTRACT

This invention relates to a quality control method for improving histoprocessing, as well as a device for measuring the thickness of an organic tissue sample suited for use in the method.

### BACKGROUND OF THE INVENTION

1. Field of the Invention

This invention relates to a quality control method for improving histoprocessing of organic tissue samples, to a device for measuring the thickness of such a sample, and to the use of such device in histology.

2. Related Technology

In the field of pathology, it is customary to prepare slice-shaped tissue samples for processing, staining, and histopathologic analysis. One practice of examining fresh or fixed patient tissue samples is referred to as “grossing” and involves preparation of relatively thick tissue samples, i.e. samples in the order of 0.5 mm to 4 mm thickness are considered the norm. In certain cases, the specimens may have a thickness up to 10 mm.

The standardization of cassettes of an internal limit of 5 mm thickness is considered to be the physical limit of tissue thickness for routine histoprocessing. During the grossing process, representative tissue sample slices are taken from various sites of the tissue for subsequent histoprocessing steps such as: fixing in formalin, dehydrating, clearing and ultimately impregnating in paraffin wax. This enables paraffin sections to be cut and stained for microscopical examination and diagnosis.

The thickness and uniformity of the tissue sample slice determine histoprocessing time and quality of the histoprocessed tissue sample slice for diagnosis.

The current practice in routine grossing is to rely on the skill of an experienced pathologist or pathology assistant for estimating the tissue sample slice thickness and maintaining its uniform thickness during the cutting process. This process is done manually which involves grasping the tissue with a pair of forceps at a distal end, holding the tissue down at the point of contact with the forceps, and then cutting. Alternatively, the tissue is sandwiched between single pronged forceps or fingers. The pathologist then cuts through the tissue without any physical guide for ensuring a desired thickness and its uniformity. As is evident, this practice normally does not produce tissue sample slices of a predetermined uniform thickness. The process is further complicated when cutting very soft tissues in a fresh state or fatty tissue, which are extremely difficult to cut to a predetermined thickness even for the most experienced pathologist.

Lack of uniformity in the thickness of a tissue sample block results in poor histoprocessing rendering a subsequent microscopic diagnosis difficult if not impossible. It is, therefore, essential to cut a tissue sample slice having a uniform—preferably less than 3 mm—thickness in order to ensure consistent histoprocessing quality that does not jeopardize subsequent microscopic diagnosis.

In U.S. Pat. No. 6,513,803 issued Feb. 4, 2003, Morales et al. disclose a grossing board having one or more tissue receiving depressions formed therein. A knife guiding assembly is provided to guide a knife or scalpel blade along a surface of the grossing board to cut the tissue sample to a predetermined thickness defined by the depth of the respective depression. While the grossing board facilitates the preparation of some tissue samples, tissues that are very soft, smooth and slippery, such as those having a high fat content, are difficult to hold in the receiving depressions of the grossing board. Furthermore, the receiving depressions are difficult to clean resulting in an increased risk of cross contamination between different samples.

In U.S. Patent Application Publication US 2004/0061269 A1 (Apr. 1, 2004), Morales discloses a pathology grossing tool comprising first and second tine arrays for engaging and holding the tissue. The first and second tine arrays define a slice thickness there between when a slice is prepared by cutting parallel to the first and second tine array using the tine arrays as guides for a cutting tool. Unfortunately, by engaging the tissue with the tine array the tissue structure is distorted in the vicinity of the tines and, further, the tissue is compressed between the tines resulting in an uneven slice thickness after cutting. Furthermore, improper engagement of the tines with the tissue, in particular, towards the end of the cutting process, allows movement of the tissue sample due to shearing stress induced by the moving cutting tool also resulting in an uneven thickness of the sample slice.

### SUMMARY OF THE INVENTION

The invention provides a method and a device for use in the method by which the thickness of a sample can be checked easily in order to facilitate histoprocessing.

The present invention thus provides, in a first aspect, a quality control method for improving histoprocessing of an organic tissue sample comprising:

a) setting a maximum organic tissue sample thickness value;

b) setting histoprocessing parameters as a function of the set maximum tissue sample thickness value;

c) grossing the tissue sample to be processed;

d) setting the set maximum thickness value as a threshold value on a thickness measurement device;

e) measuring the thickness of the tissue sample using the thickness measurement device, said device comprising a contactless measuring unit and comprising a unit for automatically indicating whether the thickness of the cut sample is above or below the set threshold value; and,

f) in the case that the thickness of the cut sample is below the set threshold value, processing the sample using the parameters determined in b).

The invention also provides a device for measuring and/or controlling the thickness of a sample, comprising:

a) a base plate for placing the sample, and

b) a thickness measuring unit for measuring the top of the surface of a sample to the base plate.

Finally, the use of a device of the invention in histology pertains to a further aspect of the invention.

### BRIEF DESCRIPTION OF THE DRAWINGS

There follows a detailed description of preferred embodiments of the invention, to be read together with the accompanying drawings, wherein:

FIG. 1 is a schematic representation of a device according to the invention; and,

FIG. 2 is a more detailed depiction of the measurement station.

### DETAILED DESCRIPTION OF THE INVENTION

The invention relates to a quality control method for improving histoprocessing of an organic tissue sample. Histoprocessing implies all the processes which have to be carried out on a sample after grossing and prior to further investigation. The processing steps involve

treatment with formalin or saline solution to stabilise the tissue

dehydration of the sample (usually with alcohol)

removal of lipids by a solvent

infiltration and impregnation of the tissue with a supporting medium.

The inventive method comprises first setting a maximum organic tissue sample thickness value. This is a theoretical maximum thickness value.

For histoprocessing, the samples may vary from 1 mm to 10 mm in thickness. Typically, the samples are from 2 mm to 5 mm in thickness. For this thickness range, standard biopsy cassettes are normally used. The maximum thickness of samples may vary up to 10 mm, in which case dedicated histoprocessing cassettes are used. The samples are typically placed in cassettes which are dipped in the appropriate solvents sequentially.

In order to facilitate the processing, the maximum thickness is set to 5 mm, preferably 3 mm, highly preferably 2 mm, and most preferably 1 mm.

According to the invention, once a maximum organic tissue sample thickness value is set, the histoprocessing parameters are set as a function of the maximum tissue thickness. The parameters comprise the type of solvent(s), the amount, the temperature, the time of solvent treatment etc. Typical solvents used for processing the samples include formalin or formalin derivatives, alcohol solutions (such as ethanol, isopropanol etc.), wax etc. These solvents are commonly used in the art and known to the skilled person.

Thus for samples having a set maximum thickness, the typical processing parameters are set to:

| | Fixation in | | | |
|---|---|---|---|---|
| | formalin or | | | Wax |
| Set | formalin | | | impregnation: |
| maximum | derivatives: | Solution of | | time, |
| thickness | time, | alcohols, time, | Vaporisation: | temperature, |
| (mm) | temperature | temperature | time, pressure | pressure |
| 1 | 15 min, 50° C. | 15 min, 50-70° C. | 1-2 min, 600 mBar | 20-25 min, 60-70° C., |
| | | | | 100-400 mBar |
| 2 | 20 min, 50° C. | 30 min, 60-70° C. | 1-2 min, 600 mBar | 45 min, 60-70° C., |
| | | | | 100-500 mBar |
| 3 | 30 min, 50° C. | 80 min, 60-70° C. | 1-2 min, 600 mBar | 70-75 min, 60-70° C., |
| | | | | 100-500 mBar |
| 4 | 40 min, 50° C. | 130-140 min, | 1-2 min, 600 mBar | 85-90 min, 60-70° C., |
| | | 60-70° C. | | 100-500 mBar |
| 5 | 60 min, 50° C. | 150 min, 60-70° C. | 1-2 min, 600 mBar | 120 min, 60-70° C., |
| | | | | 100-500 mBar |
| 6 | 70 min, 50° C. | 190-200 min, | 1-2 min, 600 mBar | 130-140 min, 60-70° C., |
| | | 60-70° C. | | 100-500 mBar |

The solution of alcohols may be replaced by a solution of ethanol followed by a solution of isopropanol. The parameters are then set as shown in the table below:

| | Fixation in | | | | |
|---|---|---|---|---|---|
| | formalin or | | | | |
| Set | formalin | | | Vaporisation: | |
| maximum | derivatives: | Ethanol: | Isopropanol: | time, | Wax: time, |
| thickness | time, | time, | time, | pressure, | temperature, |
| (mm) | temperature | temperature | temperature | temperature | pressure |
| 1 | 15 min, | 15 min, 60-70° C. | 16 min, 60-70° C. | 1-2 min, 600 mBar, | 23 min, 60-70° C. |
| | 50° C. | | | 70° C. | |
| 2 | 20 min, | 20 min, 60-70° C. | 28 min, 60-70° C. | 1-2 min, 600 mBar, | 45 min, 60-70° C. |
| | 50° C. | | | 70° C. | |
| 3 | 30 min, | 25 min, 60-70° C. | 72 min, 60-70° C. | 1-2 min, 600 mBar, | 74 min, 60-70° C. |
| | 50° C. | | | 70° C. | |
| 4 | 40 min, | 30 min, 60-70° C. | 102 min, 60-70° C. | 1-2 min, 600 mBar, | 89 min, 60-70° C. |
| | 50° C. | | | 70° C. | |
| 5 | 60 min, | 40 min, 60-70° C. | 112 min, 60-70° C. | 1-2 min, 600 mBar, | 120 min, 60-70° C. |
| | 50° C. | | | 70° C. | |
| 6 | 70 min, | 50 min, 60-70° C. | 132 min, 60-70° C. | 1-2 min, 600 mBar, | 135 min, 60-70° C. |
| | 50° C. | | | 70° C. | |

Alternatively, a solution of alcohols followed by isopropanol may be used after fixation. This is preferably used in the case of fatty tissues. The parameters used are shown below:

| | Fixation in | | | | |
|---|---|---|---|---|---|
| | formalin or | | | | |
| Set | formalin | Solution of | | Vaporisation: | |
| maximum | derivatives: | alcohols: | Isopropanol: | time, | Wax: time, |
| thickness | time, | time, | time, | pressure, | temperature, |
| (mm) | temperature | temperature | temperature | temperature | pressure |
| 3 | 30 min, | 50 min, 60-70° C. | 20 min, 60-70° C. | 1-2 min, 600 mBar | 74 min, 60-70° C. |
| | 50° C. | | | | |
| 4 | 40 min, | 105 min, 60-70° C. | 25 min, 60-70° C. | 1-2 min, 600 mBar, | 89 min, 60-70° C. |
| | 50° C. | | | | |
| 5 | 60 min, | 120 min, 60-70° C. | 30 min, 60-70° C. | 1-2 min, 600 mBar, | 120 min 60-70° C. |
| | 50° C. | | | 70° C. | |
| 6 | 70 min, | 140 min, 60-70° C. | 60 min, 60-70° C. | 1-2 min, 600 mBar | 135 min 60-70° C. |
| | 50° C. | | | | |

Grossing is then carried out, according to the inventive method. This may be done using any suitable tools. Because it is very difficult for the skilled person to cut an even thin slice of tissue and, because it is also difficult for the skilled person to estimate the exact thickness of the sample, it is important to measure the thickness of the sample by reliable means. This is achieved in the invention by using a thickness measurement device comprising a contactless measuring unit and comprising a unit for automatically indicating whether the thickness of the cut sample is above or below the set threshold value.

It is important, however, that prior to measuring, the set maximum thickness value is set as a threshold value on the thickness measurement device.

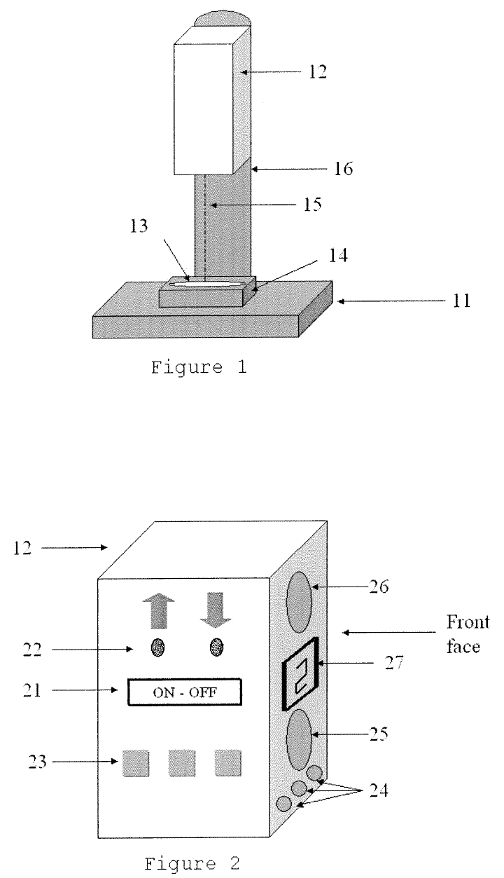

A thickness measurement device which can be used in the present method is illustrated in FIG. 1.

Referring to FIG. 1, the tissue sample measurement device pertaining to the invention comprises a base plate 11 for placing the sample 13 and a contactless thickness measuring unit for measuring the distance from the top of the surface of the sample to the base plate.

The thickness measuring unit present in the device of the invention is contactless, which means that no interaction of a user with the sample is required to carry out the measurement. This helps in preventing contamination of the sample. Contactless measuring units may include distance measuring transducers such as laser, ultrasound, or optical transducers, etc. It is preferably a laser-based measuring device. Thus, according to a preferred embodiment, the laser-based measuring unit comprises a laser 15, the direction of which is perpendicular to the base plate. Furthermore, the laser-based measuring unit preferably comprises a laser sensor having a resolution of 50 microns to 100 microns, preferably 80 microns.

In the preferred embodiment shown in FIG. 1, the thickness measuring unit is placed above the base plate 11. The measuring unit is preferably contained in a measurement station 12 which may be adjustable in height above the sample 13. To adjust the height of the measurement station, said measurement station is preferably fixed on a movable support 16.

The device of the invention preferably comprises a device 21 for switching the device on and off. Furthermore, it may comprise a device 22 for presetting a threshold thickness value. This can be useful, for example, when a sample should not go beyond or below a certain thickness. Thus, depending on the subsequent application of the sample, the threshold thickness may be increased or decreased.

Preferably, the device also comprises a device for indicating transgression of a preset threshold thickness value. The device for indicating transgression may be visual and/or acoustic, for example. For instance, this may be in the form of a red light, a flashing light, a beep, acoustic signals with different frequencies etc. Preferably, as shown in FIG. 2, the measuring station is provided with two lights 25 and 26, one green and one red, which are activated when the sample is below or above the threshold thickness value respectively.

In the method of the invention, so long as the measured thickness of the cut sample is below the set threshold value, the processing of the samples using the histoprocessing parameters determined above is carried out.

Regarding the device, the measuring station may further comprise a unit for displaying the thickness value 27. The thickness value may be a predetermined threshold thickness value, as is the case when used in the method of the invention. In another embodiment, it may be an absolute value as measured by the measuring unit.

Preferably, the device is such that a carrier 14 for the sample is present. The carrier may be any type of container capable of holding the sample. Preferably, the carrier is a standard cassette commonly used in histology.

Furthermore, the inventive device may comprise preset offset values. Thus, when a specific carrier is used, the measurement unit is pre-calibrated such that the measurement of the sample itself is accurate. The device may comprise a plurality of such preset offset values. These may be selectively activated by means on the measurement station 23. In the embodiment shown in FIG. 2, there are 3 possible preset offset values, for example, small, medium, or large. The selected preset offset value may be displayed on the measuring station. Typically, this is done by using lights 24 to indicate which value is selected.

This is particularly useful when using, for example, standard carriers having predetermined dimensions. Indeed, in the field of histology in particular, commercially available cassettes are obtainable in varying standard sizes. The invention allows the device to be preset to these cassettes, such that the thickness measuring unit offsets the dimension of the cassette to allow precise measurement of the sample.

Thus, in order to ensure accurate measurement, it is preferable that the measuring unit of the device may be calibrated. This may be done as described above, by selecting preset offset values. Alternatively, this calibration may be done automatically, prior to measurement.

Although the device is preferably operated manually, it may also be automated and may be part of an automated system.

The device of the invention is particularly suited for use in the field of histology. Indeed, by providing a method and device for measuring and/or controlling the thickness of a sample, the histology processing procedure is improved. The invention thus provides quality control benefits to the histology processing procedure. By the indication of the correct tissue thicknesses, it is possible to select faster and more appropriate processing protocols for either conventional tissue processors or rapid microwave tissue processors. Microwave tissue processors are for example known from EP 1 605 243.

The invention thus provides a method which allows the direct measurement of samples, preferably directly in the carrier in which they are to be further processed. In this way, histoprocessing can be accelerated and optimized.

### EXAMPLES

The invention is further illustrated by means of the following non-limiting examples.

### Example

The following tables (Part A and Part B) show a non-limiting example of conventional processing which can provide significant improvements due to the specimen thickness quality control of the invention:

### Part A

| | Fixation | Solution | Solution | Solution | Solution | Solution | Solution |
|---|---|---|---|---|---|---|---|
| Set | in | of 70% | of 90% | of 100% | of 100% | of 100% | of 100% |
| max | Formalin: | Alcohols: | Alcohols: | Alcohols: | Alcohols: | Alcohols: | Alcohols: |
| thickness | time, | time, | time, | time, | time, | time, | time, |
| (mm) | temperature | temperature | temperature | temperature | temperature | temperature | temperature |
| 4 | 40 min, | 10 min, | 10 min, | 15 min, | 20 min, | 20 min, | 35 min, |
| | 50° C. | 45-55° C. | 45-55° C. | 45-55° C. | 45-55° C. | 45-55° C. | 45-55° C. |
| 6 | 60 min, | 5 min, | 10 min, | 15 min, | 40 min, | 50 min, | 50 min, |
| | 50° C. | 45-55° C. | 45-55° C. | 45-55° C. | 45-55° C. | 45-55° C. | 45-55° C. |

### Part B

| Set | | | | | | |
|---|---|---|---|---|---|---|
| maximum | Xylene: | Xylene: | Xylene: | Wax: time, | Wax: time, | Wax: time, |
| thickness | time, | time, | time, | temperature, | temperature, | temperature, |
| (mm) | temperature | temperature | temperature | pressure | pressure | pressure |
| 4 | 10 min, 45-55° C. | 25 min, 45-55° C. | 35 min, 45-55° C. | 25 min, | 35 min, | 45 min, |
| | | | | 60° C., | 60° C., | 60° C., 500 mBar |
| | | | | 500 mBar | 500 mBar, | |
| 6 | 35 min, 45-55° C. | 40 min, 45-55° C. | 50 min 45-55° C. | 35 min, 45-55° C., | 50 min, | 65 min, |
| | | | | 500 mBar | 60° C., | 60° C., 500 mBar |
| | | | | | 500 mBar, | |

1. A quality control method for improving histoprocessing of an organic tissue sample, comprising:
a) setting a maximum organic tissue sample thickness value;
b) setting the histoprocessing parameters as a function of the set maximum tissue sample thickness value;
c) grossing the tissue sample to be processed;
d) setting the set maximum thickness value as a threshold value on a thickness measurement device;
e) measuring the thickness of the tissue sample using the thickness measurement device, said device comprising a contactless measuring unit and comprising a unit for automatically indicating whether the thickness of the cut sample is above or below the set threshold value; and,
f) in the case that the thickness of the cut sample is below the set threshold value, processing the sample using the parameters determined in b).

2. Method according to claim 1, wherein the maximum organic tissue sample thickness value is 5 mm.

3. Method according to claim 1, wherein the maximum organic tissue sample thickness value is 3 mm.

4. Method according to claim 1, wherein the maximum organic tissue sample thickness value is 2 mm.

5. Method according to claim 1, wherein the histoprocessing parameters comprise at least one parameter selected from the group consisting of the type of solvent, the amount, the temperature, and the time of solvent treatment.

6. Method according to claim 1, wherein the unit for automatically indicating whether the thickness of the cut sample is above or below the set threshold value provides at least one of a visual and an acoustic indication.

7. Method according to claim 1, wherein the thickness measurement device comprises a laser-based measurement device.

8. Method according to claim 7, wherein the measurement device comprises a laser sensor having a resolution of 50 microns to 100 microns.

9. Method according to claim 8, wherein the resolution is 80 microns.

10. Measurement device for measuring the thickness of an organic tissue sample in a method according to claim 1, said device comprising
i) a base plate for placing the sample; and,
ii) a contactless thickness measuring unit for measuring the distance from the top of the surface of the sample to the base plate.
